(19) United States
(12) Patent Application Publication
(10) Pub. No.: US 2016/0303114 A1
(43) Pub. Date: Oct. 20, 2016

(54) PHARMACEUTICAL COMPOSITIONS CONTAINING VANOXERINE AND P450 INHIBITORS AND METHODS OF TERMINATING ACUTE EPISODES OF CARDIAC ARRHYTHMIA, RESTORING NORMAL SINUS RHYTHM, PREVENTING RECURRENCE OF CARDIAC ARRHYTHMIA AND MAINTAINING NORMAL SINUS RYTHYM IN MAMMALS THROUGH ADMINISTRATION OF SAID COMPOSITIONS

(71) Applicant: Laguna Pharmaceuticals, Inc., La Jolla, CA (US)

(72) Inventor: Arthur M. Brown, La Jolla, CA (US)

(73) Assignee: Laguna Pharmaceuticals, Inc., La Jolla, CA (US)

(21) Appl. No.: 14/781,289

(22) PCT Filed: Apr. 25, 2014
(86) PCT No.: PCT/US2014/035544

§ 371 Date: Sep. 29, 2015

Related U.S. Application Data
(60) Provisional application No. 61/816,214, filed on Apr. 26, 2013.
(60) Provisional application No. 61/904,949, filed on Nov. 15, 2013.

(51) Int. Cl. A61K 31/495; G01N 33/49; A61K 9/00; A61K 9/20; A61K 9/48; A61K 36/752

(52) U.S. Cl. CPC A61K 31/495; A61K 9/4858; A61K 9/4866; A61K 9/485; A61K 36/752; A61K 9/0053; A61K 9/2054; A61K 9/2013; A61K 9/2018; A61K 9/2009; G01N 33/49; G01N 2800/52

(57) ABSTRACT

Compositions comprising vanoxerine (GBR 12909) and a P450 inhibitor, including compositions of vanoxerine and one or more P450 inhibitors, processes for their preparation thereof, and methods of using the same for treatment of cardiac arrhythmias.

### FIELD OF THE INVENTION

Presently disclosed embodiments are related to pharmaceutical compositions of vanoxerine and P450 inhibitors and processes for the preparation thereof. Presently disclosed embodiments particularly relate to pharmaceutical compositions that include vanoxerine and at least one P450 inhibitor in combination with one or more diluents, disintegrants, binders and/or lubricants.

### BACKGROUND

Vanoxerine (1-[2-[bis(4-fluorophenyl)methoxy]ethyl]-4-(3-phenylpropyl)piperazine), its manufacture and/or certain pharmaceutical uses thereof are described in U.S. Pat. No. 4,202,896, U.S. Pat. No. 4,476,129, U.S. Pat. No. 4,874,765, U.S. Pat. No. 6,743,797 and U.S. Pat. No. 7,700,600, as well as European Patent EP 243,903 and PCT International Application WO 91/01732, each of which is incorporated herein by reference in its entirety.

Vanoxerine has been used for treating cocaine addiction, acute effects of cocaine, and cocaine cravings in mammals, as well as dopamine agonists for the treatment of Parkinsonism, acromegaly, hyperprolactinemia and diseases arising from a hypofunction of the dopaminergic system. (See U.S. Pat. No. 4,202,896 and WO 91/01732). Vanoxerine has also been used for treating and preventing cardiac arrhythmia in mammals. (See U.S. Pat. No. 6,743,797 and U.S. Pat. No. 7,700,600).

Atrial flutter and/or atrial fibrillation (AF) are the most commonly sustained cardiac arrhythmias in clinical practice, and are likely to increase in prevalence with the aging of the population. Currently, AF affects more than 1 million Americans annually, represents over 5% of all admissions for cardiovascular diseases and causes more than 80,000 strokes each year in the United States. In the US alone, AF currently afflicts more than 2.3 million people. By 2050, it is expected that there will be more than 12 million individuals afflicted with AF. While AF is rarely a lethal arrhythmia, it is responsible for substantial morbidity and can lead to complications such as the development of congestive heart failure or thromboembolism. Currently available Class I and Class III anti-arrhythmic drugs reduce the rate of recurrence of AF, but are of limited use because of a variety of potentially adverse effects, including ventricular proarrhythmia. Because current therapy is inadequate and fraught with side effects, there is a clear need to develop new therapeutic approaches.

Current first line pharmacological therapy options for AF include drugs for rate control. Despite results from several studies suggesting that rate control is equivalent to rhythm control, many clinicians believe that patients are likely to have better functional status when in sinus rhythm. Further, being in AF may introduce long-term mortality risk, where achievement of rhythm control may improve mortality.

Ventricular fibrillation (VF) is the most common cause associated with acute myocardial infarction, ischemic coronary artery disease and congestive heart failure. As with AF, current therapy is inadequate and there is a need to develop new therapeutic approaches.

Although various anti-arrhythmic agents are now available on the market, those having both satisfactory efficacy and a high margin of safety have not been obtained. For example, anti-arrhythmic agents of Class I, according to the classification scheme of Vaughan-Williams (“Classification of antiarrhythmic drugs,” Cardiac Arrhythmias, edited by: E. Sandoe, E. Flensted-Jensen, K. Olesen; Sweden, Astra, Sodertalje, pp 449-472 (1981)), which cause a selective inhibition of the maximum velocity of the upstroke of the action potential (Vₘₐₓ) are inadequate for preventing ventricular fibrillation because they shorten the wave length of the cardiac action potential, thereby favoring re-entry. In addition, these agents have problems regarding safety, i.e. they cause a depression of myocardial contractility and have a tendency to induce arrhythmias due to an inhibition of impulse conduction. The CAST (coronary artery suppression trial) study was terminated while in progress because the Class I antagonists had a higher mortality than placebo controls. β-adrenergenic receptor blockers and calcium channel (I_{Ca}) antagonists, which belong to Class II and Class IV, respectively, have a defect in that their effects are either limited to a certain type of arrhythmia or are contraindicated because of their cardiac depressant properties in certain patients with cardiovascular disease. Their safety, however, is higher than that of the anti-arrhythmic agents of Class I.

Prior studies have been performed using single dose administration of flecainide or propafenone (Class I drugs) in terminating atrial fibrillation. Particular studies investigated the ability to provide patients with a known dose of one of the two drugs so as to self-medicate should cardiac arrhythmia occur. P. Alboni, et al., “Outpatient Treatment of Recent-Onset Atrial Fibrillation with the ‘Pill-in-the-Pocket’ Approach,” NEJM 351; 23 (2004); L. Zhou, et al., “‘A Pill in the Pocket’ Approach for Recent Onset Atrial Fibrillation in a Selected Patient Group,” Proceedings of UCLA Healthcare 15 (2011). However, the use of flecainide and propafenone has been criticized as including candidates having structural heart disease and thus providing patients likely to have risk factors for stroke who should have received antithrombotic therapy, instead of the flecainide or propafenone. NEJM 352:11 (Letters to the Editor) (Mar. 17, 2005). Similarly, the use of warfarin concomitantly with propafenone was criticized.

Anti-arrhythmic agents of Class III are drugs that cause a selective prolongation of the action potential duration (APD) without a significant depression of the maximum upstroke velocity (Vₘₐₓ). They therefore lengthen the save length of the cardiac action potential increasing refractories, thereby antagonizing re-entry. Available drugs in this class are limited in number. Examples such as sotalol and amiodarone have been shown to possess interesting Class III properties (Singh B. N., Vaughan Williams E. M., “A Third Class of Anti-Arrhythmic Action: Effects on Atrial and Ventricular Intracellular Potentials and other Pharmacological Actions on Cardiac Muscle of MJ 1999 and AH 3747,” (Br. J. Pharmacol 39:675-689 (1970), and Singh B. N., Vaughan Williams E. M., “The Effect of Amiodarone, a New Anti-Anginal Drug, on Cardiac Muscle,” Br. J. Pharmacol 39:657-667 (1970)), but these are not selective Class III agents. Sotalol also possesses Class II (β-adrenergic blocking) effects which may cause cardiac depression and is contraindicated in certain susceptible patients.

Amiodarone also is not a selective Class III antiarrhythmic agent because it possesses multiple electrophysiological actions and is severely limited by side effects. (Nademanee, K., “The Amiodarone Odyssey,” J. Am. Coll. Cardiol. 20:1063-1065 (1992)). Drugs of this class are expected to be effective in preventing ventricular fibrillation. Selective Class III agents, by definition, are not considered to cause myocardial depression or an induction of arrhythmias due to inhibition of conduction of the action potential as seen with Class I antiarrhythmic agents.

Class III agents increase myocardial refractoriness via a prolongation of cardiac action potential duration (APD). Theoretically, prolongation of the cardiac action potential can be achieved by enhancing inward currents (i.e. Na+ or Ca²+ currents; hereinafter I_{Na} and I_{Ca}, respectively) or by reducing outward repolarizing potassium K+ currents. The delayed rectifier (I_{K}) K+ current is the main outward current involved in the overall repolarization process during the action potential plateau, whereas the transient outward (Iₜₒ) and inward rectifier (I_{KI}) K+ currents are responsible for the rapid initial and terminal phases of repolarization, respectively.

Cellular electrophysiologic studies have demonstrated that I_{K} consists of two pharmacologically and kinetically distinct K+ current subtypes, I_{Kr} (rapidly activating and deactivating) and I_{Ks} (slowly activating and deactivating). (Sanguinetti and Jurkiewicz, “Two Components of Cardiac Delayed Rectifier K+Current. Differential Sensitivity to Block by Class III Anti-Arrhythmic Agents,” J Gen Physiol 96:195-215 (1990)). I_{Kr} is also the product of the human ether-a-go-go gene (hERG). Expression of hERG cDNA in cell lines leads to production of the hERG current which is almost identical to I_{Kr} (Curran et al., “A Molecular Basis for Cardiac Arrhythmia: hERG Mutations Cause Long QT Syndrome,” Cell 80(5):795-803 (1995)).

Class III anti-arrhythmic agents currently in development, including d-sotalol, dofetilide (UK-68,798), almokalant (H234/09), E-4031 and methanesulfonamide-N-[1′-6-cyano-1,2,3,4-tetrahydro-2-naphthalenyl)-3,4-dihydro-4-hydroxyspiro[2H-1-benzopyran-2, 4′-piperidin]-6yl], (+)-, monochloride (MK-499) predominantly, if not exclusively, block I_{Kr}. Although amiodarone is a blocker of I_{Ks} (Balser J. R. Bennett, P. B., Hondeghem, L. M. and Roden, D. M. “Suppression of time-dependent outward current in guinea pig ventricular myocytes: Actions of quinidine and amiodarone,” Circ. Res. 69:519-529 (1991)), it also blocks I_{Na} and I_{Ca}, effects thyroid function, as a nonspecific adrenergic blocker, acts as an inhibitor of the enzyme phospholipase, and causes pulmonary fibrosis (Nademanee, K., “The Amiodarone Odessey.” J. Am. Coll. Cardiol. 20:1063-1065 (1992)).

Reentrant excitation (reentry) has been shown to be a prominent mechanism underlying supraventricular arrhythmias in man. Reentrant excitation requires a critical balance between slow conduction velocity and sufficiently brief refractory periods to allow for the initiation and maintenance of multiple reentry circuits to coexist simultaneously and sustain AF. Increasing myocardial refractoriness, by prolonging APD, prevents and/or terminates reentrant arrhythmias. Most selective Class III antiarrhythmic agents currently in development, such as d-sotalol and dofetilide predominantly, if not exclusively, block I_{Kr}, the rapidly activating component of I_{K} found both in atria and ventricle in man.

Since these I_{Kr} blockers increase APD and refractoriness both in atria and ventricle without affecting conduction per se, theoretically they represent potential useful agents for the treatment of arrhythmias like AF and VF. These agents have a liability in that they have an enhanced risk of proarrhythmia at slow heart rates. For example, torsade de pointes, a specific type of polymorphic ventricular tachycardia which is commonly associated with excessive prolongation of the electrocardiographic QT interval, hence termed “acquired long QT syndrome,” has been observed when these compounds are utilized (Roden, D. M., “Current Status of Class III Antiarrhythmic Drug Therapy,” Am J. Cardiol, 72:44B-49B (1993)). The exaggerated effect at slow heart rates has been termed “reverse frequency-dependence” and is in contrast to frequency-independent or frequency-dependent actions. (Hondeghem, L. M., “Development of Class III Antiarrhythmic Agents,” J. Cardiovasc. Cardiol. 20 (Suppl. 2):S17-S22). The pro-arrhythmic tendency led to suspension of the SWORD trial when d-sotalol had a higher mortality than placebo controls.

The slowly activating component of the delayed rectifier (I_{Ks}) potentially overcomes some of the limitations of I_{Kr} blockers associated with ventricular arrhythmias. Because of its slow activation kinetics, however, the role of I_{Ks} in atrial repolarization may be limited due to the relatively short APD of the atrium. Consequently, although I_{Ks} blockers may provide distinct advantage in the case of ventricular arrhythmias, their ability to affect supraventricular tachyarrhythmias (SVT) is considered to be minimal.

Another major defect or limitation of most currently available Class III anti-arrhythmic agents is that their effect increases or becomes more manifest at or during bradycardia or slow heart rates, and this contributes to their potential for proarrhythmia. On the other hand, during tachycardia or the conditions for which these agents or drugs are intended and most needed, they lose most of their effect. This loss or diminishment of effect at fast heart rates has been termed “reverse use-dependence” (Hondeghem and Snyders, “Class III antiarrhythmic agents have a lot of potential but a long way to go: Reduced Effectiveness and Dangers of Reverse use Dependence,” Circulation, 81:686-690 (1990); Sadanaga et al., “Clinical Evaluation of the Use-Dependent QRS Prolongation and the Reverse Use-Dependent QT Prolongation of Class III Anti-Arrhythmic Agents and Their Value in Predicting Efficacy,” Amer. Heart Journal 126:114-121 (1993)), or “reverse rate-dependence” (Bretano, “Rate dependence of class III actions in the heart,” Fundam. Clin. Pharmacol. 7:51-59 (1993); Jurkiewicz and Sanguinetti, “Rate-Dependent Prolongation of Cardiac Action Potentials by a Methanesulfonanilide Class III Anti-Arrhythmic Agent: Specific Block of Rapidly Activating Delayed Rectifier K+ current by Dofetilide,” Circ. Res. 72:75-83 (1993)). Thus, an agent that has a use-dependent or rate-dependent profile, opposite that possessed by most current class III anti-arrhythmic agents, should provide not only improved safety but also enhanced efficacy.

Vanoxerine has been indicated for treatment of cardiac arrhythmias. Indeed, certain studies have looked at the safety profile of vanoxerine and stated that no side-effects should be expected with a daily repetitive dose of 50 mg of vanoxerine. (U. Sogaard, et. al., “A Tolerance Study of Single and Multiple Dosing of the Selective Dopamine Uptake Inhibitor GBR 12909 in Healthy Subjects,” International Clinical Psychopharmacology, 5:237-251 (1990)). However, Sogaard, et. al. also found that upon administration of higher doses of vanoxerine, some effects were seen with regard to concentration difficulties, increase systolic blood pressure, asthenia, and a feeling of drug influence, among other effects. Sogaard, et. al. also recognized that there were unexpected fluctuations in serum concentrations with regard to these healthy patients. While they did not determine the reasoning, control of such fluctuations may be important to treatment of patients.

Further studies have looked at the ability of food to lower the first-pass metabolism of lipophilic basic drugs, such as vanoxerine. (S. H. Ingwersen, et. al., “Food Intake Increases the Relative Oral Bioavailability of Vanoxerine,” Br. J. Clin. Pharmac; 35:308-130 (1993)). However, no methods have been utilized or identified for treatment of cardiac arrhythmias in conjunction with the modulating effects of food intake.

In view of the problems associated with current anti-arrhythmic agents, there remains a need for an effective treatment of cardiac arrhythmias in mammals with a vanoxerine compound and a P450 inhibitor to maintain therapeutic plasma level of vanoxerine. The newly discovered formulations preferably use a minimal number of excipients and use pharmaceutical grade excipients that are inexpensive, readily available, and that facilitate cost-effective manufacture on a commercial scale for the treatment of among cardiac arrhythmias. Furthermore, these compositions are further utilized in the methods disclosed herein for the treatment of cardiac arrhythmias utilizing vanoxerine and a P450 inhibitor provide for improved treatment procedures and methods for treating patients suffering events of cardiac arrhythmia.

### SUMMARY

Embodiments of the present disclosure relate to novel compositions of vanoxerine and a P450 inhibitor. In particular, vanoxerine and a P450 inhibitor are admixed with various excipients to formulate a solid dose of vanoxerine. In certain embodiments, the solid dose is in tablet form; in other embodiments, it is in capsule form and in further other embodiments, the formulation is suitable for buccal, sublingual, nasal, oral, topical, rectal and parenteral administration.

An embodiment is a pharmaceutical composition for the treatment of cardiac arrhythmia, in unit dosage form comprising vanoxerine, in an amount of from about 20-50% of the composition by weight; a P450 inhibitor from about 1-30% of the composition by weight, a diluent in an amount of from about 20-60% of the composition by weight; a binder in an amount of from about 10-25% of the composition by weight; a disintegrant in an amount of from about 1-5% of the composition by weight; a flowing agent from about 0.2-0.4% of the composition by weight; and a lubricant from about 0.2-0.4% of the composition by weight.

A further embodiment is a method of minimizing variability of plasma level concentrations for treatment of cardiac arrhythmia with vanoxerine comprising: determining a target plasma level; administering a first dose of a drug comprising vanoxerine to a patient; measuring plasma levels of said patient; modifying said first dose of a drug by modifying the amount of vanoxerine; and administering a subsequent dose to said patient.

One embodiment is a method for modulating plasma level concentrations in a patient being treated for cardiac arrhythmia comprising: administering a first dose of vanoxerine; measuring the physiological concentration of vanoxerine; calculating an effective dose of vanoxerine and a P450 inhibitor to modulate the plasma level concentration; and administering the effective dose of vanoxerine and P450 inhibitor.

One embodiment is a method for maintaining a pre-determined plasma level comprising: administering a first dose of vanoxerine; measuring the physiological concentration of vanoxerine and one or more metabolites; administering a second dosage of vanoxerine in conjunction with a P450 inhibitor; measuring the physiological concentration and one or more metabolites; modifying the dosage of vanoxerine and P450 inhibitor based on the differences between the plasma level after the first administration and the second administration; and administering at least a third dosage of vanoxerine and P450 inhibitor to maintain a pre-determined plasma level.

One embodiment is a method for administering vanoxerine for treatment of cardiac arrhythmia comprising: administering a first dose of vanoxerine to a patient; measuring the physiological concentration of vanoxerine in the patient; calculating an effective dose of vanoxerine and a P450 inhibitor to modify the calculated plasma level concentration; and administering the effective dose of vanoxerine and P450 inhibitor.

One embodiment is a method for maintaining a pre-determined plasma concentration of vanoxerine in a mammal comprising: determining a pre-determined target plasma concentration in the mammal; administering a first dose of vanoxerine to said mammal; measuring the plasma concentration of vanoxerine in the mammal; determining an appropriate subsequent dose of vanoxerine to be taken with a concomitant administration of a P450 inhibitor; and administering said subsequent dose of vanoxerine and P450 inhibitor to said mammal.

A further embodiment is pharmaceutical composition for the treatment of cardiac arrhythmia, in unit dosage form comprising vanoxerine, in an amount of from about 20-50% of the composition by weight; a P450 inhibitor from about 1-30% of the composition by weight, a diluent in an amount of from about 20-60% of the composition by weight; a binder in an amount of from about 10-25% of the composition by weight; a disintegrant in an amount of from about 1-5% of the composition by weight; a flowing agent from about 0.2-0.4% of the composition by weight; and a lubricant from about 0.2-0.4% of the composition by weight.

An additional embodiment therefore comprises a pharmaceutical composition comprising vanoxerine and one or more P450 inhibitor, suitable for administration to a mammal for the treatment of cardiac arrhythmias.

An additional aspect of the present disclosure includes processes for the preparation of vanoxerine formulations. In particular, the processes involve preparation of a solid dosage form of vanoxerine and a P450 inhibitor, preferably by wet mixing vanoxerine and excipients with water, followed by drying and milling of the granulated mixture.

An additional aspect of the present disclosure include methods of treatment of a disease or disorder in a subject in need thereof comprising administering to the subject a therapeutically effective amount of vanoxerine and a P450 inhibitor to provide therapeutic levels of the vanoxerine compound of the presently disclosed embodiments.

An additional aspect of the present disclosure include methods of treatment of cardiac arrhythmias comprising administering vanoxerine and a P450 inhibitor to a mammal, such as a human, to provide therapeutic plasma levels of the vanoxerine compound.

An additional aspect of the present disclosure comprises methods for terminating acute episodes of cardiac arrhythmia, such as atrial fibrillation or ventricular fibrillation, in a mammal, such as a human, by administering to that mammal at least an effective amount of vanoxerine and a P450 inhibitor to terminate an acute episode of cardiac arrhythmia.

An additional aspect of the present disclosure is directed to a method for restoring normal sinus rhythm in a mammal, such as a human, exhibiting cardiac arrhythmia by administering at least an effective amount of vanoxerine and a P450 inhibitor to restore normal sinus rhythm.

An additional aspect of the present disclosure is directed to a method for maintaining normal sinus rhythm in a mammal, such as a human, by administering at least an effective amount of vanoxerine and a P450 inhibitor to maintain normal sinus rhythm in a mammal that has experienced at least one episode of cardiac arrhythmia.

Further embodiments of the present disclosure relate to methods for treating cardiac arrhythmias comprising: determining a physiological concentration for vanoxerine for treating cardiac arrhythmias; administering a first dose of vanoxerine and one or more P450 inhibitor; measuring the physiological concentration of vanoxerine; modifying the dosage of vanoxerine and one or more P450 inhibitor based on the measurement of the physiological concentration; and administering a further dosage of vanoxerine and one or more P450 inhibitor.

Further embodiments of the present disclosure relate to methods for treating cardiac arrhythmias comprising: administering a first dose of vanoxerine to a patient; measuring the physiological concentration of vanoxerine; administering a second dosage of vanoxerine in conjunction with one or more P450 inhibitor; measuring the physiological concentration of vanoxerine; modifying the dosage of vanoxerine and one or more P450 inhibitor based on the differences between the physiological concentration after the first administration and the second administration; and administering at least a third dosage of vanoxerine and one or more P450 inhibitor to maintain a pre-determined plasma level.

Further embodiments of the present disclosure relate to methods for treating cardiac arrhythmias comprising: administering a first dose of vanoxerine to a patient; measuring the physiological concentration of vanoxerine and one or more metabolites of vanoxerine; administering a second dosage of vanoxerine in conjunction with one or more P450 inhibitors; measuring the physiological concentration of vanoxerine and/or one or more metabolites of vanoxerine; modifying the dosage of vanoxerine and one or more P450 inhibitors based on the differences between the physiological concentration after the first administration and the second administration to achieve a pre-determined physiological concentration in the patient, and administering the modified dose to a patient.

An additional aspect of the present disclosure includes a method for treating cardiac arrhythmias comprising administering a first dose of a pharmaceutical composition comprising vanoxerine and one or more P450 inhibitor to a patient; measuring the physiological level of vanoxerine in the patient; calculating the required dose to provide for a plasma level of about 20 to about 100 ng/ml, 1 hour post administration, and administering the calculated dose. A further embodiment provides that the dose provides an effective plasma level concentration of between 20 and 200 ng/ml at 1 to 4 hours post administration.

Other aspects of the present disclosure include methods of treatment of a disease or disorder in a subject in need thereof comprising administering to the subject a therapeutically effective amount of vanoxerine and one or more P450 inhibitor to maintain therapeutic physiological levels of vanoxerine, measuring the physiological levels of vanoxerine in the subject; adjusting the dosage of vanoxerine and/or one or more P450 inhibitor; and administering a subsequent dose of vanoxerine and one or more P450 inhibitor based on the adjusted dosage.

Other aspects of the present disclosure include methods of treatment of cardiac arrhythmias comprising administering vanoxerine and one or more P450 inhibitor to maintain therapeutic physiological levels of the vanoxerine compound; measuring the physiological levels in the subject; adjusting the dosage of vanoxerine and one or more P450 inhibitor to meet a pre-determined physiological level; and administering a subsequent dose of vanoxerine and one or more P450 inhibitor based on the adjusted dosage.

Other aspects of the present disclosure comprise methods for terminating acute episodes of cardiac arrhythmia, such as atrial fibrillation or ventricular fibrillation, in a mammal, such as a human, by administering to that mammal at least an effective amount of vanoxerine and one or more P450 inhibitor to terminate an acute episode of cardiac arrhythmia; measuring the physiological levels in the subject; adjusting the dosage of vanoxerine and one or more P450 inhibitor; and administering a subsequent dose of vanoxerine and one or more P450 inhibitor based on the adjusted dosage.

Another aspect of the present disclosure is directed to a method for restoring normal sinus rhythm in a mammal, such as a human, exhibiting cardiac arrhythmia by administering at least an effective amount of vanoxerine and one or more P450 inhibitor selected from the group consisting of: CYP3A4, CYP2C8, CYP2E1, and CYP2D6, and combinations thereof, to restore normal sinus rhythm; measuring the physiological levels or vanoxerine in the subject; adjusting the dosage of vanoxerine and one or more P450 inhibitor based on the measured physiological concentration; and administering a subsequent dose of vanoxerine and one or more P450 inhibitor based on the adjusted dosage.

Another aspect of the present disclosure is directed to a method for maintaining normal sinus rhythm in a mammal, such as a human, by administering at least an effective amount of vanoxerine and one or more P450 inhibitor to maintain normal sinus rhythm in a mammal that has experienced at least one episode of cardiac arrhythmia; measuring the plasma levels in the subject; adjusting the dosage of vanoxerine and one or more P450 inhibitor; and administering a subsequent dose of vanoxerine and one or more P450 inhibitor based on the adjusted dosage.

Another aspect of the present disclosure is directed to a method for preventing a recurrence of an episode of cardiac arrhythmia in a mammal, such as a human, by administering to that mammal at least an effective amount of vanoxerine and one or more P450 inhibitor to prevent a recurrence of cardiac arrhythmia; measuring the plasma levels in the subject; adjusting the dosage of vanoxerine and one or more P450 inhibitor; and administering a subsequent dose of vanoxerine and one or more P450 inhibitor based on the adjusted dosage.

Other methods of the present disclosure are directed to methods for modulation of vanoxerine Cₘₐₓ and tₘₐₓ with regard to a particular patient, wherein a first effective dose of a drug comprising vanoxerine is administered; measuring the physiological concentration subsequent to administration; determining the Cₘₐₓ and/or tₘₐₓ for the patient; determining further effective doses to modulate the Cₘₐₓ and/or tₘₐₓ through the inclusion of one or more P450 inhibitors to a pre-determined physiological level, and administering at least one further effective dose of vanoxerine.

An additional aspect of the present disclosure is directed to a method for preventing a recurrence of an episode of cardiac arrhythmia in a mammal, such as a human, by administering to that mammal at least an effective amount of vanoxerine and a P450 inhibitor to prevent a recurrence of cardiac arrhythmia.

### DETAILED DESCRIPTION OF PREFERRED EMBODIMENTS

All references cited herein are hereby incorporated by reference in their entirety.

As used herein, the term “about” is intended to encompass a range of values ±10% of the specified value(s). For example, the phrase “about 20” is intended to encompass ±10% of 20, i.e. from 18 to 22, inclusive.

As used herein, the term “vanoxerine” refers to vanoxerine and pharmaceutically acceptable salts thereof.

As used herein, the term “pharmaceutically acceptable” refers to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable benefit/risk ratio.

As used herein, the term “subject” refers to a warm blooded animal such as a mammal, preferably a human or a human child, which is afflicted with, or has the potential to be afflicted with one or more diseases and conditions described herein.

As used herein, “therapeutically effective amount” refers to an amount which is effective in reducing, eliminating, treating, preventing or controlling the symptoms of the herein-described diseases and conditions. The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment.

As used herein, “unit dose” means a single dose which is capable of being administered to a subject, and which can be readily handled and packaged, remaining as a physically and chemically stable unit dose comprising either vanoxerine or a pharmaceutically acceptable composition comprising vanoxerine.

As used herein, “CYP3A4” means the cytochrome P450 3A4 protein, which is a monooxygenase that is known for its involvement in drug metabolism.

As used herein, “CYP2C8” means the cytochrome P450 2C8 protein, which is a monooxygenase that is known for its involvement in drug metabolism.

As used herein, “CYP2E1” means the cytochrome P450 2E1 protein, which is a monooxygenase that is known for its involvement in drug metabolism.

As used herein, “CYP2D6” means the cytochrome P450 2D6 protein, which is a monooxygenase that is known for its involvement in drug metabolism.

As used herein, “P450” means cytochrome P450 superfamily, which is a diverse group of enzymes facilitating oxidation of organics within the body.

As used herein, “inhibitor” is typically used with one of the cytochrome P450 proteins, meaning a compound that works as a substrate inhibitor and inhibits the production of the particular P450 protein.

As used herein, “administering” or “administer” refers to the actions of a medical professional or caregiver, or alternatively self-administration by the patient.

As used herein, “antianginal” means any drug used in the treatment of angina pectoris, or chest pain due to ischemia of the heart muscle.

The term “steady state” means wherein the overall intake of a drug is fairly in dynamic equilibrium with its elimination.

As used herein, a “pre-determined” plasma level or other physiological tissue or fluid and refers to a concentration of vanoxerine at a given time point. Typically, a pre-determined level will be compared to a measured level, and the time point for the measured level will be the same as the time point for the pre-determined level. In considering a pre-determined level with regard to steady state concentrations, or those taken over a period of hours, the pre-determined level is referring to the mean concentration taken from the area under the curve (AUC), as the drug increases and decreases in concentration in the body with regard to the addition of a drug pursuant to intake and the elimination of the drug via bodily mechanisms.

Cardiac arrhythmias include atrial, junctional, and ventricular arrhythmias, heart blocks, sudden arrhythmic death syndrome, and include bradycardias, tachycardias, re-entrant, and fibrillations. These conditions, including the following specific conditions: atrial flutter, atrial fibrillation, multifocal atrial tachycardia, premature atrial contractions, wandering atrial pacemaker, supraventricular tachycardia, AV nodal reentrant tachycardia, junctional rhythm, junctional tachycardia, premature junctional contraction, premature ventricular contractions, ventricular bigeminy, accelerated idioventricular rhythm, monomorphic ventricular tachycardia, polymorphic ventricular tachycardria, and ventricular fibrillation, and combinations thereof are all capable of severe morbidity and death if left untreated. Methods and compositions described herein are suitable for the treatment of these and other cardiac arrhythmias.

Interestingly, studies have identified that human subjects have significant variability with regard to the metabolism of vanoxerine. Vanoxerine, is susceptible to metabolism by CYP3A4 among other known P450 cytochromes. Accordingly, the bioavailability of a given dose of vanoxerine is impacted by certain P450 cytochromes. In particular, studies have identified that human subjects have variability with regard to metabolism which is predicted to be based on CYP3A4 and other P450 cytochromes. Typically, patients fall within one of two groups, a fast metabolism or a slow metabolism, such that the patients can be grouped with other patients and will have similar metabolic profiles for a given dose of vanoxerine. Patients in the fast metabolism group respond differently to vanoxerine than patients in the slow metabolism group with regard to Cₘₐₓ, tₘₐₓ, and AUC plasma concentrations as well as the half-life. Accordingly, it is possible to define whether a given patient is a fast or a slow metabolizer and predict their pharmacokinetic response to vanoxerine. Accordingly, determination of the patient's status within the fast or slow metabolic group can be utilized for improving efficacy and treatment of a patient.

Additionally, patients fall within a gradient within the slow and fast metabolism groups. Accordingly, there exists, even within the groupings, a continuum that provides that some people are faster or slower metabolizers even within the groups. Additional factors also play into the variability with regard to patient populations. Accordingly, when providing efficacious treatment for termination of cardiac arrhythmias, in some embodiments, it is important to determine or recognize where the patient falls within the spectrum of vanoxerine bioavailability, and provide a dose of vanoxerine that will be efficacious for that patient while also maximizing the safety profile of the drug.

Vanoxerine also has a moderately low oral bioavailability as a result of incomplete absorption and substantial first pass metabolism, from CYP3A4 and other p450 inhibitors. Vanoxerine is primarily eliminated from the body in urine, bile, and feces. Indeed, a substantial amount of the drug is expelled unabsorbed into the feces. Additionally, pharmacokinetic parameters from tests in dogs suggest that there is a slow Tₘₐₓ of about 3 hours, low systemic bioavailability (23%) and slow elimination from the plasma (T_{1/2} of 22 hours). However, the long half-life of the drug may actually be utilized to minimize the continuous or regular dosing of the drug.

Vanoxerine, once in the body, metabolizes, at least partially, into at least five metabolites including: M01, M02, M03, M04, and M05. Accordingly, it is advantageous to modify and calibrate not only the measured amounts of vanoxerine, but also to measure and calibrate the metabolites thereof. Accordingly, a method comprises a first dose of vanoxerine, measuring the concentrations of vanoxerine and/or one or more of metabolites M01, M02, M03, M04, and M05. Once the concentration of vanoxerine and/or one or more of the metabolites is known at a given time point, subsequent treatment may be modified based on the results. The modification will be based on the goal for the Cₘₐₓ for vanoxerine and/or one or more of the metabolites. The Cₘₐₓ and tₘₐₓ for vanoxerine and any of the metabolites impacts the efficacy of treatment.

In view of the susceptibility to metabolism by numerous P450s including CYP3A4, CYP2C8, CYP2E1, and CYP2D6 among other known P450 cytochromes, certain advantages exist by eliminating first pass metabolism by enzymatic catalysts and increasing the bioavailability with regard to a given dose of the vanoxerine compound. Accordingly, the bioavailability of a given dose of a vanoxerine compound can be modified by blocking and/or having a P450 antagonist to prevent the metabolism of the vanoxerine compound before it has the ability to act on its intended target. This provides for either purely increased bioavailability per a given dose, or the opportunity to minimize a dose of the vanoxerine compound and still provide an efficacious dose of the drug compound.

Similarly, studies with co-administration of vanoxerine with a particular inhibitor for CYP2C8 and CYP2E1, and CYP2D6, like CYP3A4, will affect first pass metabolism. Because all patients are different, the concentration of the particular P450 and the particular efficiency of first pass metabolism in a patient is different, blocking one or a number of P450's can provide for modification of Cₘₐₓ and tₘₐₓ and allow for modification of treatments based on the ability to improve efficacy in a patient.

Certain mammalian subjects react differently to P450 inhibitors, this may be in part due to the variability of mammalian subjects in their ability to metabolize P450 inhibitors, or based on the plasma levels of the particular P450 in the individual. However, in view of the metabolic variability within individuals, Applicant has invented compositions and methods of treatment of cardiac arrhythmias to administer a unit dose comprising vanoxerine to heighten the dose response, modulate the maximum plasma levels, modulate the time to maximum plasma levels, increase the duration of a heightened plasma level, and other mechanisms that are effective for treating episodes of cardiac arrhythmia.

Methods of administering vanoxerine to patients based on their P450 metabolism profile can be utilized to improve the efficacy of vanoxerine in the treatment of cardiac arrhythmias. For example, in the case of a fast metabolism, vanoxerine is metabolized by first pass metabolism and thereby limiting the effective Cₘₐₓ. Modulation of the Cₘₐₓ then comprises a method of giving a first dosage, measuring the concentration of vanoxerine in the patient in tissue, blood, plasma, or other fluids at a given point subsequent to the administration of the first dose, adjusting the dosage of vanoxerine and further comprising a CYP3A4, a CYP2C8, CYP2E1, or a CYP2D6 inhibitor, or combinations thereof to modify the Cₘₐₓ; and administering a further dose at the adjusted dosage rate provides for improved therapeutic levels for the patient.

Other methods, however, may choose to modify the dosage by including a P450 inhibitor with the first and subsequent dosage. For example, a method of treatment comprising administering a first dose comprising vanoxerine and a P450 inhibitor, measuring the physiological levels (in plasma or other fluid or tissues); modifying the dose by changing the amount of P450 inhibitor; and the modified dose comprising vanoxerine and the P450 inhibitor is administered to the patient.

Similarly, a first dose may comprise vanoxerine and a P450 inhibitor; measuring the plasma levels of vanoxerine; determining the appropriate concentrations of vanoxerine and P450 inhibitor based on the measured physiological levels; modifying both the amounts of vanoxerine and P450 inhibitor in a subsequent dose for administration; and administering the modified dose.

Similarly, other methods may particularly include a CYP3A4, CYP2C8, CYP2E1, or a CYP2D6 inhibitor. Similarly, compositions that act as an inhibitor for a broad spectrum of P450's may be utilized to inhibit these and other P450s that are acting on the vanoxerine drug.

Modifying doses and limiting the amount of vanoxerine to be administered is important for safe and effective treatment of arrhythmic patients. Studies have questioned whether sustained, and/or chronic use of vanoxerine is suitable for mammalian patients. Preliminary studies have suggested that daily use of a drug over 7, 10, and 14 days may lead to increased heart rate and systolic blood pressure when taking concentrations of 75, 100, 125, and 150 mg of vanoxerine a day. However, control and prevention of events of cardiac arrhythmia are important to these patients to prevent future recurrences and the deleterious effects and morbidity.

Indeed, control and prevention of events of cardiac arrhythmia are important to these patients to prevent future recurrences and the deleterious effects and morbidity. One issue is that cardiac arrhythmia is a progressive disease and patients who suffer from a first cardiac arrhythmia are pre-disposed to suffering from additional episodes of cardiac arrhythmia. Any cardiac arrhythmia involves risk with regard to mortality and morbidity, and so terminating the cardiac arrhythmia in a timely and safe manner is a critical need for these patients. Therefore preventing further arrhythmic events is paramount to preventing this risk.

Additional concerns for patients who have suffered from cardiac arrhythmia are compounding heart disease, as well as angina pectoris as well as other heart pain, chest pain, and other complications. Typically, concomitant use of an atrial fibrillation drug with a number of other drugs is contraindicated because of any number of interactions between the two drugs. However, certain drugs may establish a beneficial co-administration with vanoxerine wherein the concomitant administration of vanoxerine and at least one additional drug (p450 inhibitor) for treatment of cardiac arrhythmia allows for modified vanoxerine dosing. For example, the combination allows for administration of reduced doses of vanoxerine to maintain normal sinus rhythm,

One suitable P450 inhibitor is grapefruit juice and/or other citrus juices. Studies have shown that grapefruit juice, taken in conjunction with certain medications, significantly increases the bioavailability of the drug as it acts as an inhibitor on the metabolism of the CYP3A4 enzyme to prevent the metabolism of the drug compound. Studies have shown that 6′, 7′-dihydroxybergamottin, a furanocoumarin, may be responsible for the inhibition of the CYP3A4 enzyme. (See Edwards, Clinical Pharma & Thera., 65:237-244, 1999). However, other studies have postulated that it is the grapefruit flavonoids, furanocoumarins and/or related compounds from grapefruit that provide these inhibition properties, and that the effect may be seen from more than one component as found in the grapefruit juice. (See Ho and Saville, J. Pharm Pharmaceut. Sci.; 4(3):217-227, 2001).

There are other suitable compounds that also show CYP3A4 inhibitory properties. For example, ritonavir, an antiretroviral drug originally used to treat HIV infection, has shown to inhibit CYP3A4 when in use as a concomitant therapy. The drug is now sporadically used for antiviral activity, but is administered for its use as a concomitant inhibitor of certain cytochrome P450 isoforms, including CYP3A4.

Dexamethasone is another drug that is known to have certain CYP3A4 activity, and its concomitant use may be utilized to inhibit the CYP3A4 enzyme to prevent the initial breakdown of vanoxerine indicated herein. Similarly, erythromycin, a macrolide antibiotic with a wide antimicrobial spectrum is known to be taken concomitantly with certain statin drugs where the statin has an increased activity. Concomitant use of erythromycin with vanoxerine provides for a mechanism to increase the bioavailability of the piperazine compound, where the erythromycin serves as a CYP3A4 antagonist allowing for the increased bioavailability of the vanoxerine compound.

Warfarin, an anticoagulation drug is frequently administered to patients who suffer from cardiac arrhythmias because of other underlying health issues. Warfarin is a CYP3A4 substrate. Accordingly, with co-administration with vanoxerine, the warfarin is likely to serve as the CYP3A4 inhibitor. In some cases, it may be advantageous to only utilize the warfarin as the CYP3A4 inhibitor, yet in other cases an additional inhibitor may be appropriate. However, warfarin is contraindicated for many arrhythmic drugs because of these very properties.

Accordingly, use of the grapefruit or other citrus juice, flavonoids, furanocoumarins, ritonavir, dexamethasone, erythromycin, warfarin, and combinations thereof, in combination with vanoxerine, can serve to provide for increased bioavailability of vanoxerine. This allows for reduced dosing of vanoxerine by increasing the bioavailability of the dose through limiting first pass metabolism.

Similar agents are known with regard to inhibition of P450's such as CYP2E1, CYP2C8, and CYP2D6. Accordingly, the use of these and other agents identified with regard to CPY3A4 in particular, may be suitable for the inhibition of one or a combination of these P450 agents. Alternatively, narrowly targeted agents are often preferred to particularly inhibit only one of the P450 proteins, and the use of these narrowly targeted agents is preferred with regard to their use in pharmaceutical compositions and the methods described herein.

Suitable methods for the use of vanoxerine and a P450 inhibitor include combination therapies, taking vanoxerine and independently taking a P450 inhibitor, and formulating a drug product comprising both vanoxerine and a P450 inhibitor. Accordingly, the use of the concomitant therapy is used to modify the effects of vanoxerine where such compound is given in an effective amount to impact cardiac arrhythmias.

Accordingly, some embodiments are compositions comprising vanoxerine and a P450 inhibitor. The combination of the vanoxerine compound and the active compound(s) making up the P450 inhibitor may be combined into a single dose form, i.e. a single composition administered as a single drug, or given in two separate doses, the vanoxerine compound and a P450 inhibitor.

In certain preferred embodiments, an oral formulation provides a composition comprising vanoxerine, a P450 inhibitor, blocking CYP3A4, CYP2C8, CYP2E1, or CYP2D6, either individually, or combinations thereof, and optionally a diluent such as lactose, a binder such as microcrystalline cellulose, a disintegrant such as croscarmellose sodium, a flowing agent such as colloidal silicon dioxide, and a lubricant such as magnesium stearate.

The excipients are selected to ensure the delivery of a consistent amount of vanoxerine and to maintain plasma levels of the vanoxerine compound through the co-administration of the P450 inhibitor, in a convenient unit dosage form and to optimize the cost, ease and reliability of the manufacturing process. All excipients must be inert, organoleptically acceptable, and compatible with vanoxerine. The excipients used in a solid oral formulation commonly include fillers or diluents, binders, disintegrants, lubricants, antiadherents, glidants, wetting and surface active agents, colors and pigments, flavoring agents, sweeteners, adsorbents, and taste-maskers.

Diluents are typically added to a small amount of the active drug to increase the size of the tablet. A suitable diluent for use in the inventive compositions is lactose, which exists in two isomeric forms, alpha-lactose or beta-lactose, and can be either crystalline or amorphous. Various types of lactose include spray dried lactose monohydrate (such as Super-Tab™), alpha-lactose monohydrate (such as Fast Flo®), anhydrous alpha-lactose, anhydrous beta-lactose, and agglomerated lactose. Other diluents include sugars, such as compressible sugar NF, dextrose excipient NF, and dextrates NF. A preferred diluent is lactose monohydrate (such as Fast Flo®). Other preferred diluents include microcrystalline cellulose (such as Avicel® PH, and Ceolus™) and microfine cellulose (such as Elcema®).

Suitable diluents also include starch and starch derivatives. Starches include native starches obtained from wheat, corn, rice and potatoes. Other starches include pregelatinized starch NF, and sodium starch glycolate NF. Starches and starch derivatives can also function as disintegrants. Other diluents include inorganic salts, including, but not limited to, dibasic calcium phosphate USP (such as Di-Tab® and Emcompress®), tribasic calcium phosphate NF (such as Tri-Tab® and Tri-Cafos®), and calcium sulfate NF (such as Compactrol®). Polyols such as mannitol, sorbitol, and xylitol may also serve as diluents. Many diluents can also function both as disintegrants and as binders, and these additional properties should be taken into account when developing particular formulations.

Disintegrants may be included to break larger particles, such as tablets, granules, beads, nonpareils and/or dragees, into smaller particles comprising the active pharmaceutical ingredient and, optionally, other excipients which may facilitate dissolution of the active ingredient and/or enhance bioavailability of the active ingredient. Starch and starch derivatives, including cross-linked sodium salt of a carboxymethyl ether of starch (such as sodium starch glycolate NF, Explotab®, and Primogel®) are useful disintegrants. A preferred disintegrant is cross-linked sodium carboxymethyl cellulose (such as Croscarmellose Sodium NF, Ac-Di-Sol®). Other suitable disintegrants include, but are not limited to, cross-linked polyvinylpyrrolidone (such as Crospovidone NF) and microcrystalline cellulose (such as Avicel® PH).

Binders may also be used as an excipient, particularly during wet granulation processes, to agglomerate the active pharmaceutical ingredient and the other excipients. In all formulation, whether prepared by wet or dry granulation, a particular binder is generally selected to improve powder flow and/or to improve compactibility. Suitable binders include, but are not limited to, cellulose derivatives, such as microcrystalline cellulose NF, methylcellulose USP, carboxymethycellulose sodium USP, hydroxypropyl methylcellulose USP, hydroxyethyl cellulose NF, and hydroxypropyl cellulose NF. Other suitable binders include polyvidone, polyvinyl pyrrolidone, gelatin NF, natural gums (such as acacia, tragacanth, guar, and pectin), starch paste, pregelatinized starch NF, sucrose NF, corn syrup, polyethylene glycols, sodium alginate, ammonium calcium alginate, magnesium aluminum silicate and polyethylene glycols.

Lubricants may be used, particularly in tablet formulations, to prevent sticking of the ingredients and/or dosage form to the punch faces and to reduce friction during the compression stages. Suitable lubricants include, but are not limited to, vegetable oils (such as corn oil), mineral oils, polyethylene glycols (such as PEG-4000 and PEG-6000), salts of stearic acid (such as calcium stearate and sodium stearyl fumarate), mineral salts (such as talc), inorganic salts (such as sodium chloride), organic salts (such as sodium benzoate, sodium acetate, and sodium oleate) and polyvinyl alcohols. A preferred lubricant is magnesium stearate.

In preferred embodiments, vanoxerine generally comprises from about 20-50% by weight of the pharmaceutical composition, more preferably from about 25-40% and most preferably from about 30-35%.

In preferred embodiments, a P450 inhibitor generally comprises from about 1-50% by weight of the pharmaceutical composition, more preferably from about 1-20% and from about 1-10% by weight when formulated in a solid oral formulation or liquid capsule, or the like. The P450 inhibitor may also be taken as a separate dose in a separate dosage form, such as a volume of citrus juice taken concomitantly with a solid oral dosage form of vanoxerine. Such amounts include about 1-16 ounces of a juice, more preferably about 2-8 ounces and most preferably about 4-8 ounces of juice.

In effectively treating cardiac arrhythmia, it is necessary in some circumstances to provide for a certain plasma level concentration of vanoxerine. Plasma level concentrations are modified by the compositions and methods described herein. As patients have variability with regarding to their first pass metabolism of vanoxerine, modification of the metabolic pathways by blocking certain metabolic agents can provide advantages for administering vanoxerine to a patient. Effective plasma level concentrations, taken at a time point of 1 hour post administration are about 5 to about 1000 ng/ml. In alternative embodiments, plasma level concentrations at 1 hour post administration are about 10 to about 400 ng/ml, or about 20 to about 200 ng/ml, or about 20 to about 150 ng/ml, or about 25 to about 125 ng/ml or about 40 to about 100 ng/ml, and about 60 to about 100 ng/ml.

Furthermore, it is advantageous in some embodiments to provide for a certain dose, or a maximum dose at a given time point after administration of the vanoxerine to safely and effectively treat the cardiac arrhythmia. Accordingly, modification of Cₘₐₓ and tₘₐₓ is appropriate to maintain consistent plasma level concentrations for a particular patient. Cₘₐₓ taken at a time point of 1 hour post administration are about 5 to about 1000 ng/ml. In alternative embodiments, physiological concentrations, as measured in the plasma at a time of 1 hour post administration are about 20 to about 400 ng/ml, or about 20 to about 200 ng/ml, or about 25 to about 150 ng/ml or about 40 to about 125 ng/ml, or about 60 to about 100 ng/ml. Conversely tₘₐₓ is appropriately reached at about 1 hour post administration. In other embodiments, tₘₐₓ is appropriately reached at about 30 minutes, or about 90 minutes, or about 120 minutes, or about 240 minutes post administration.

Suitable methods for treatment of cardiac arrhythmias include various dosing schedules which may be administered by any technique capable of introducing a pharmaceutically active agent to the desired site of action, including, but not limited to, buccal, sublingual, nasal, oral, topical, rectal and parenteral administration. Dosing may include single daily doses, multiple daily doses, single bolus doses, slow infusion injectables lasting more than one day, extended release doses, IV or continuous dosing through implants or controlled release mechanisms, and combinations thereof. These dosing regimens in accordance with the method allow for the administration of the vanoxerine in an appropriate amount to provide an efficacious level of the compound in the blood stream or in other target tissues. Delivery of the compound may also be through the use of controlled release formulations in subcutaneous implants or transdermal patches.

Preferably, in oral formulations, embodiments comprising vanoxerine and a P450 inhibitor also comprises a diluent which is lactose monohydrate, a binder which is microcrystalline cellulose; a disintegrant which is a cross-linked sodium carboxymethyl cellulose; a flowing agent which is colloidal silicon dioxide, and a lubricant which is magnesium stearate. Suitable amounts of each excipient may be determined empirically by one skilled in the art considering such factors as the particular mode of administration (e.g. oral, sublingual, buccal, etc.), amount of active ingredient (e.g. 50 mg, 60 mg, 80 mg, 100 mg, 150 mg, etc.), particular patient (e.g. adult human, human child, etc.) and dosing regimen (e.g. once a day, twice a day, etc.).

In certain preferred embodiments, the inventive compositions may contain lactose monohydrate (e.g. Fast Flo® #316) from about 30-60% of the composition by weight, more preferably from about 35-50% and most preferably from about 40-45%.

In certain preferred embodiments, the inventive compositions may contain microcrystalline cellulose (e.g. Avicel® PH 102) from about 5-30% by weight of the composition, more preferably from about 10-25% and most preferably from about 15-20% by weight,

In certain preferred embodiments, the inventive compositions may contain cross-linked sodium carboxymethyl cellulose (e.g. Ac-Di-Sol®) from about 0.1-10% by weight of the composition, more preferably from about 0.5-5% and most preferably from about 1-3% by weight,

In certain preferred embodiments, the inventive compositions may contain colloidal silicon dioxide (e.g. Aerosil® A-200) from about 0.02 to about 1% by weight of the composition, more preferably form about 0.1 to about 0.6% and most preferably from about 0.2-0.4% by weight.

In certain preferred embodiments, the inventive compositions may contain magnesium stearate from about 0.02 to about 1% by weight of the composition, more preferably form about 0.1 to about 0.6% and most preferably from about 0.2-0.4% by weight.

Tablets may also be formulated in a manner known in the art so as to give a sustained release of vanoxerine. Such tablets may, if desired, be provided with enteric coatings by known method, for example by the use of cellulose acetate phthalate. Suitable binding or granulating agents are e.g., gelatine, sodium carboxymethylcellulose, methylcellulose, polyvinylpyrrolidone or starch gum. Talc, colloidal silicic acid, stearin as well as calcium and magnesium stearate or the like can be used as anti-adhesive and gliding agents.

Tablets may also be prepared by wet granulation and subsequent compression. A mixture containing vanoxerine and at least one diluent, and optionally a part of the disintegrating agent, is granulated together with an aqueous, ethanolic or aqueous-ethanolic solution of the binding agents in appropriate equipment, then the granulate is dried. Thereafter, other preservative, surface acting, dispersing, disintegrating, gliding and anti-adhesive additives can be mixed to the dried granulate and the mixture can be compressed to tablets or capsules.

Tablets may also be prepared by the direct compression of the mixture containing the active ingredient together with the needed additives. If desired, the tablets may be transformed to dragees by using protective, flavoring and dyeing agents such as sugar, cellulose derivatives (methyl- or ethylcellulose or sodium carboxymethylcellulose), polyvinylpyrrolidone, calcium phosphate, calcium carbonate, food dyes, aromatizing agents, iron oxide pigments and the like which are commonly used in the pharmaceutical industry.

For the preparation of capsules or caplets, vanoxerine, or vanoxerine and/or an anti-anginal compound as disclosed herein, or a pharmaceutically acceptable salt thereof, and the desired additives may be filled into a capsule, such as a hard or soft gelatin capsule. The contents of a capsule and/or caplet may also be formulated using known methods to give sustained release of the active compound.

Liquid oral dosage forms of vanoxerine and vanoxerine and/or an anti-anginal compound as disclosed herein, or a pharmaceutically acceptable salt thereof, may be an elixir, suspension and/or syrup, where the compound is mixed with a non-toxic suspending agent. Liquid oral dosage forms may also comprise one or more sweetening agent, flavoring agent, preservative and/or mixture thereof.

In particular, the process comprises the steps of: (a) dry blending of vanoxerine, a P450 inhibitor, and one or more excipients to form a dry mixture; (b) wetting the dry mixture with water, preferably with purified water, to form a wet granulation mixture; (c) drying the wet granulation mixture to form a dried granulation mixture; (d) milling the dried granulation mixture to form a milled granulation mixture; (e) mixing a lubricant in the milled granulation mixture to give a final blended mixture; (f) preparing the final blended mixture in a solid dosage form suitable for oral administration.

In certain preferred embodiments, the final blended mixture is compressed into tablets. In other preferred embodiments, the final blended mixture is enclosed in a capsule. The particular tablets, capsules, or other forms may be modified to provide various amounts of vanoxerine and/or a P450 inhibitor to provide for the ability to modify the amounts of the vanoxerine and P450 in view of the requirements of the methods described herein.

Specifically, in step (a), vanoxerine and the P450 inhibitor are blended with all excipients in the final formulation, other than the lubricant. In particular, vanoxerine and the P450 inhibitor are thoroughly dry blended with the diluent(s), disintegrant(s) and binder to form a uniform dry mixture. Blenders appropriate for large scale dry blending include twin shell blenders, double cone blenders, and ribbon blenders. Ribbon blenders have the advantage of being used in continuous-production procedures. High-speed, high shear mixers may also be used and offer the advantage of shorter mixing times. The dry mixture may also be granulated, milled into a fine powder, passed through a mesh screen, or micronized, if necessary. Preferably, the dry blending was performed in high shear granulators.

The resulting dry mixture is then wetted with a wetting agent to form a wet granulation mixture in step (b). The wetting agent is typically added over time, usually from about 1 to about 15 minutes, with continuous mixing. Typically, the wetting agent is added to the blender used in the dry blending step. Preferably the wet granulation is carried out in a high shear granulator. In certain embodiments, the wetting agent is an aqueous-based solution. Preferably, the wetting agent is water without any additional solvents, and in particular, without organic solvents. More preferably, the water is purified water.

The type and amount of wetting agent, rate of addition of wetting agent, and the mixing time influences the structure of the granules. The different types of granules, such as pendular, funicular, capillary, etc., can be manipulated to achieve the desired density, porosity, texture and dissolution pattern of the granules, which in turn, determines the compressibility, hardness, disintegration and consolidation characteristics of the dried mixture.

The wet granulation mixture is then dried in step (c) to form a dried granulation mixture with an appropriate moisture content. In certain embodiments, the drying means include a fluid bed or tray dryers. Fluid bed drying yield shorter drying times, in the range from 1 to 3 hours, while tray drying averages 10 to 13 hours. Preferably, the wet granulation mixture is dried in a fluid bed, for preferably about 1-3 hours. Fluid bed drying has the added advantages of better temperature control and decreased costs. The method of drying, drying time, and moisture content are critical to avoid decomposition, chemical migration, and other adverse physical characteristics of dried mixture which can affect the dosage form performance.

The dried granulation mixture is subsequently milled in step (d) to form a milled granulation mixture. The particle size of the dried granulation mixture is reduced to achieve an appropriate particle size distribution for the subsequent processes. In certain embodiments, milling is achieved using a high shear impact mill (such as Fitzpatrick) or a low shear screening mill (such as Comil). The dried granulation mixture may also be screened to select the desired granule size.

In the next step (e), the lubricant was blended with the dried granulation mixture to give a final blended mixture. In certain embodiments, a V blender or bin blenders are used. A preferred blender is a V-shell PK blender. A gentle blending is preferred, such that each granule covered with the lubricant, while minimizing the breaking up of the granules. Increased breaking of the granules results in fine powder, or “fines”. A high fine content results in variations of weight and density during compression into a tablet, as well as increases the need for cleaning of the compression machinery.

The final blended mixture is then prepared in a solid dosage form suitable for oral administration. Solid dosage forms include tablets, capsules, pills, troches, cachets, and the like. In one embodiment, the final blended mixture is compressed into a tablet. The compression machinery typically contains two steel punches within a steel die cavity. The tablet is formed when pressure is exerted on the dried granulation mixture by the punches in the cavity, or cell.

Tableting machines include single-punch machines, rotary tablet machines, gravity feed, and powder assisted machines. Preferably, gravity feed or powder assisted machines are used. Rotary machines operating at high speeds suitable for large-scale production include double rotary machines and single rotary machines. Tablets can also include sugar-coated tablets, film-coated tablets, enteric-coated tablets, multiple-compressed tablets, controlled-release tablets, tablets for solution, effervescent tablets or buccal and sublingual tablets.

Compressed tablets may be characterized by a number of specifications, including diameter size, shape, thickness, weight, hardness, friability, disintegration time, and dissolution characteristics. The tablets preferably have weights, friability and dissolution rates in accordance with USP standards.

In other embodiments, the final blended mixture is enclosed in capsules, preferably hard gelatin capsules. The hard gelatin capsules are commercially available, and are generally made from gelatin, colorants, optionally an opacifying agent such as titanium dioxide, and typically contain 12-16% water. The hard capsules can be prepared by filling the longer end of the capsule with the final blended mixture, and slipping a cap over the top using mG2, Zanasi, or Hofliger and Karg (H&K) machines.

In an alternative embodiment, the present invention provides for a process of preparing a solid dose form of vanoxerine and a P450 inhibitor by dry mixing vanoxerine and the P450 inhibitor with the excipients. In certain embodiments, the mixture is compressed into a tablet. In other embodiments, the mixture is encapsulated.

In particular, the process comprises the steps of: (a) dry blending of vanoxerine, a P450 inhibitor and one or more excipients to form a dry mixture; (b) mixing a lubricant in the dry mixture to give a final blended mixture; (c) preparing the final blended mixture in a solid dosage form suitable for oral administration.

Specifically, in step (a), vanoxerine and a P450 inhibitor are blended with all excipients in the final formulation, other than the lubricant. Preferably, vanoxerine and the P450 inhibitor are thoroughly dry blended with the diluent(s), disintegrant(s) and a binder to form a uniform dry mixture. Blenders appropriate for large scale dry blending include twin shell blenders, double cone blenders, V blenders or bin blenders. A preferred blender is a V-shell PK blender. High-speed, high shear mixers may also be used. The dry mixture may also be granulated, milled into a fine powder, passed through a mesh screen, or micronized, if necessary.

In the next step (b), the lubricant was blended with the dry mixture to give a final blended mixture. In certain embodiments, a V blender or bin blenders are used. A preferred blender is a V-shell PK blender.

The final blended mixture is then prepared in a solid dosage form suitable for oral administration. Solid dosage forms include tablets, capsules, pills, troches, cachets, and the like. In one embodiment, the final blended mixture is compressed into a tablet. In another embodiment, the final blended mixture is enclosed in capsules, preferably hard gelatin capsules.

For rectal administration, a suitable composition containing vanoxerine and vanoxerine and/or an anti-anginal compound as disclosed herein, or a pharmaceutically acceptable salt thereof, may be prepared in the form of a suppository. In addition to the active ingredient, the suppository may contain a suppository mass commonly used in pharmaceutical practice, such as Theobroma oil, glycerinated gelatin or a high molecular weight polyethylene glycol.

For parenteral administration, a suitable composition of vanoxerine and vanoxerine and/or an anti-anginal compound as disclosed herein, or a pharmaceutically acceptable salt thereof, may be prepared in the form of an injectable solution or suspension. For the preparation of injectable solutions or suspensions, the active ingredient can be dissolved in aqueous or non-aqueous isotonic sterile injection solutions or suspensions, such as glycol ethers, or optionally in the presence of solubilizing agents such as polyoxyethylene sorbitan monolaurate, monooleate or monostearate. These solutions or suspensions may be prepared from sterile powders or granules having one or more carriers or diluents mentioned for use in the formulations for oral administration. Parenteral administration may be through intravenous, intradermal, intramuscular or subcutaneous injections.

Other aspects of the invention also include use methods for administering these compositions for the treatment of a disease or disorder in a subject in need thereof comprising administering to the subject a therapeutically effective amount of the compositions for the treatment of cardiac arrhythmia. A suitable method comprises administration of vanoxerine and a p450 inhibitor to a patient to achieve normal sinus rhythm.

Other aspects of the invention also include the use of these compositions in a method for achieving and/or maintaining a pre-determined plasma level of vanoxerine comprising ingesting vanoxerine and a P450 inhibitor. The P450 inhibitor and vanoxerine may be taken as a single dosage form, or taken as separate dosage forms, such as a solid dose of vanoxerine and a separate liquid P450 inhibitor. Because of the blocking of metabolic pathways, an increase in bioavailability allows for lower doses of vanoxerine to be administered to achieve efficacious Cₘₐₓ, tₘₐₓ, AUC, or a pre-determined plasma concentration, thus providing advantages of lower doses providing the same or similar efficacy in patients.

Further embodiments of the invention comprise mechanisms for modification or improvement of the efficacy, safety profile, and or modification to the administered dose of vanoxerine through the co-administration of a P450 inhibitor. Accordingly, a first administration may comprise a first dose of vanoxerine. Subsequent to the administration, the pharmacokinetic response of the patient to the vanoxerine is measured, for example, by measuring the amount of vanoxerine in the plasma of the patient. A subsequent dose of vanoxerine may then advantageously be administered concomitantly with a P450 inhibitor, such that the pharmacokinetic profile of the patient will be modified with the concomitant administration. Furthermore, the subsequent dose of vanoxerine may be modified in addition to the concomitant administration of the P450 inhibitor.

Other aspects of the disclosure provide methods for maintaining a pre-determined physiological level of vanoxerine comprising ingesting vanoxerine and a P450 inhibitor. The P450 inhibitor and vanoxerine may be taken as a single dosage form, or taken as separate dosage forms, such as a solid dose of vanoxerine and a separate liquid P450 inhibitor.

In some embodiments, a dosage of 1 mg to 1000 mg per unit dose is appropriate. Other embodiments may utilize a dosage of about 50 mg to 800 mg, or about 25 to 100 mg, or about 100 mg to about 600 mg, or about 200 to about 400 mg. Preferred doses further include about 25, 50, 75, 100, 150, 200, 300, and 400 mg doses of vanoxerine, which may be further combined with a P450 inhibitor.

Similarly, in some embodiments, a dosage of about 1 mg to about 1000 mg of the active drug component of the P450 inhibitor is utilized. Other embodiments may utilize a dosage of about 50 mg to 800 mg, or about 25 to 100 mg, or about 100 mg to about 600 mg, or about 200 to about 400 mg of the P450 inhibitor, whether the dosage form comprises both the vanoxerine and the P450 inhibitor or whether the vanoxerine is taken as a separate dosage form from the P450 inhibitor.

Physiological levels, including plasma, blood, and other body tissue concentrations are modified by the methods described herein. The administration of vanoxerine and a P450 inhibitor provides for appropriate modification of physiological concentrations. In particular regard to efficacious plasma levels in some embodiments, plasma level concentrations, taken at a time point of 1 hour post administration are about 5 to about 1000 ng/ml. In alternative embodiments, plasma level concentrations at 1 hour post administration are about 20 to about 400 ng/ml, or about 20 to about 200 ng/ml, or about 20 to about 150 ng/ml or about 25 to about 125 ng/ml, 40 to about 100 ng/ml, or about 60 to about 100 ng/ml. Furthermore, pre-determined plasma levels may be taken at other time points, and between 15-450 ng/ml between 30 minutes and 4 hours, and between 40 and 120 ng/ml between 30 minutes and 4 hours post administration.

Additionally, modification of Cₘₐₓ and tₘₐₓ is appropriate to maintain consistent plasma level concentrations. Cₘₐₓ taken at a time point of 1 hour post administration are about 5 to about 1000 ng/ml. In alternative embodiments, plasma level concentrations at 1 hour post administration are about 10 to about 400 ng/ml, or about 20 to about 200 ng/ml, or about 20 to about 150 ng/ml, or about 25 to about 125 ng/ml or about 40 to about 100 ng/ml, and about 60 to about 100 ng/ml. Conversely tₘₐₓ is appropriately reached at about 1 hour post administration. In other embodiments, tₘₐₓ is appropriately reached at about 30 minutes, or about 90 minutes, or about 120 minutes, or about 240 minutes post administration.

### EXAMPLES

The materials, methods, and examples presented herein are intended to be illustrative, and not to be construed as limiting the scope or content of the invention. Unless otherwise defined, all technical and scientific terms are intended to have their art-recognized meanings.

### Example 1

### Formulation of a 100 mg Vanoxerine Capsule

| | Amount per | Amount per |
|---|---|---|
| Components | tablet (mg) | batch (mg) |
| GBR 12909 (Vanoxerine) | 100.0 | 120.0 |
| P450 inhibitor | 10.0 | 12.0 |
| Lactose Monohydrate, NF | 121.00 | 145.20 |
| Microcrystalline Cellulose, NF | 51.00 | 61.20 |
| Croscarmellose Sodium, NF | 6.00 | 7.20 |
| Colloidal Silicon Dioxide, NF | 1.00 | 1.20 |
| Magnesium Stearate, NF | 1.00 | 1.20 |
| Total Tablet Weight | 310.0 | 348.0 |

### Example 2

### Formulation of a 200 mg Vanoxerine Capsule

| | Amount per | Amount per |
|---|---|---|
| Components | tablet (mg) | batch (mg) |
| GBR 12909 (Vanoxerine) | 200.0 | 240.0 |
| Dexamethasone | 20.0 | 24.0 |
| Lactose Monohydrate, NF | 242.00 | 290.40 |
| Microcrystalline Cellulose, NF | 102.00 | 122.40 |
| Croscarmellose Sodium, NF | 12.00 | 14.40 |
| Colloidal Silicon Dioxide, NF | 2.00 | 2.40 |
| Magnesium Stearate, NF | 2.00 | 2.40 |
| Total Tablet Weight | 620.0 | 696.0 |

### Example 3

### Formulation of a 100 mg Vanoxerine Capsule and Taken with Grapefruit Juice

| | Amount per | Amount per |
|---|---|---|
| Components | tablet (mg) | batch (mg) |
| GBR 12909 (Vanoxerine) | 100.0 | 120.0 |
| Lactose Monohydrate, NF | 121.00 | 145.20 |
| Microcrystalline Cellulose, NF | 51.00 | 61.20 |
| Croscarmellose Sodium, NF | 6.00 | 7.20 |
| Colloidal Silicon Dioxide, NF | 1.00 | 1.20 |
| Magnesium Stearate, NF | 1.00 | 1.20 |
| Total Tablet Weight | 310.0 | 348.0 |

Taken concomitantly with an 8 ounce glass of grapefruit juice.

### Example 4

15 patients are given either a dose of vanoxerine or a placebo, wherein the vanoxerine is administered in an oral tablet as vanoxerine HCL in addition to excipients as described in the embodiments herein.

All patients given vanoxerine were subsequently tested for concentrations of vanoxerine in the plasma at time points of 15 minutes, 30 minutes, 1 hour, 2 hours, and 4 hours post administration. The results identified high levels of variability between patients and a single outlier, who was particularly profiled to determine the possible cause of the high plasma concentration levels.

The outlier patient had an unusually high plasma concentration and was found to be a CYP2D6 slow metabolizer. Interesting, CYP2D6 has not previously been associated with the metabolism of vanoxerine. Accordingly, the individual patient could have received administration of ½ to ¼ the dose of other patients for the same effective plasma concentration as the majority of the patients in the study. Accordingly, co-administration of vanoxerine with a CYP2D6 antagonist may provide the same effect as a CPY2D6 slow metabolizer and allow for administration of lower doses of vanoxerine, or for patients falling within different patient profiles to have stable and increased plasma concentrations regardless of their vanoxerine metabolism profile.

### Example 5

28 patients participated in a study of vanoxerine. 25 patients took a 300 mg dose of vanoxerine and 3 patients took a placebo. Each patient gave samples before administration of their dose, and then again at nine further time points, 30 minutes after administration, 1, 2, 3, 4, 6, 8, 12, and 24 hours post administration.

TABLE 1
Concentrations ng/ml

| Time | | | | | | | Total |
|---|---|---|---|---|---|---|---|
| (h) | Vanoxerine | M03 | M04 | M01 | M02 | M05 | Metabolites |
| −15 | 1.00* | 1.00 | 1.00 | 1.00 | 1.00 | 1.00 | 1.00 |
| .5 | 25.26 | 1.02 | 10.79 | 1.93 | 1.00 | 1.30 | 12.44 |
| 1 | 70.09 | 2.46 | 49.74 | 7.51 | 1.02 | 1.88 | 60.41 |
| 2 | 104.98 | 7.08 | 82.62 | 19.65 | 1.02 | 2.59 | 111.20 |
| 3 | 81.43 | 7.21 | 75.63 | 18.68 | 1.01 | 2.14 | 102.83 |
| 4 | 54.30 | 7.54 | 63.85 | 16.42 | 1.01 | 1.45 | 88.35 |
| 6 | 32.85 | 6.59 | 48.14 | 11.48 | 1.00 | 1.22 | 66.35 |
| 8 | 24.37 | 4.92 | 38.38 | 8.98 | 1.00 | 1.21 | 52.45 |
| 12 | 15.89 | 3.98 | 26.84 | 6.30 | 1.00 | 1.05 | 37.05 |
| 24 | 8.29 | 2.32 | 13.46 | 3.66 | 1.00 | 1.01 | 19.07 |

*A quantity of (1.00) represents an amount that was below the lower limit of quantitation, which is <1.139 ng/ml vanoxerine, and <1.1141 ng/ml 17-hydroxyl vanoxerine.

TABLE 2
Standard Deviations

| Time | | | | | | | Total |
|---|---|---|---|---|---|---|---|
| (h) | Vanoxerine | M03 | M04 | M01 | M02 | M05 | Metabolites |
| −15 | 0.00 | 0.00 | 0.00 | 0.00 | 0.00 | 0.00 | 0.00 |
| .5 | 43.77 | 0.12 | 15.58 | 3.20 | 0.00 | 0.80 | 19.28 |
| 1 | 61.82 | 2.51 | 49.96 | 7.08 | 0.10 | 1.13 | 59.70 |
| 2 | 100.18 | 4.70 | 51.64 | 15.31 | 0.07 | 2.56 | 70.07 |
| 3 | 80.40 | 5.40 | 49.04 | 13.63 | 0.07 | 2.31 | 64.45 |
| 4 | 55.01 | 5.32 | 39.75 | 11.31 | 0.04 | 1.16 | 52.50 |
| 6 | 35.74 | 5.10 | 31.30 | 7.90 | 0.00 | 0.87 | 41.84 |
| 8 | 30.37 | 4.05 | 25.29 | 6.74 | 0.00 | 0.94 | 33.41 |
| 12 | 24.03 | 3.15 | 17.62 | 4.70 | 0.00 | 0.27 | 23.17 |
| 24 | 10.34 | 2.11 | 8.91 | 2.76 | 0.00 | 0.03 | 12.31 |

Table 2 shows the standard deviations from the above 25 patients receiving vanoxerine. The three patients receiving a placebo are not included in the data and all data points indicated levels of vanoxerine below the lower limit of quantitation.

Tables 1 and 2, above, show tests of 25 patients with a 300 mg dose of vanoxerine. Blood was drawn from each of the test patients before the administration of the vanoxerine, and then at 9 additional time points, one half hour after administration, then 1, 2, 3, 4, 6, 8, 12, and 24 hours subsequent to administration.

The 25 patients fall into two categories: 15 fell into a category of having the majority of time point levels that were below the average mean (as identified in Table 1) “low concentration group average,” and the remaining 10 patients had the majority of time points above the average mean “high concentration group average.”

TABLE 3
Low concentration group average:

| Time | | | | | | | Total |
|---|---|---|---|---|---|---|---|
| (h) | Vanoxerine | M03 | M04 | M01 | M02 | M05 | Metabolites |
| −15 | 1.00 | 1.00 | 1.00 | 1.00 | 1.00 | 1.00 | 1.00 |
| .5 | 16.99 | 1.00 | 12.17 | 1.52 | 1.00 | 1.37 | 13.39 |
| 1 | 40.07 | 2.78 | 56.35 | 6.76 | 1.03 | 1.73 | 66.46 |
| 2 | 42.50 | 6.48 | 74.06 | 14.09 | 1.00 | 1.30 | 94.80 |
| 3 | 31.40 | 5.36 | 59.58 | 11.38 | 1.00 | 1.14 | 76.25 |
| 4 | 24.40 | 5.91 | 51.98 | 10.34 | 1.00 | 1.05 | 68.14 |
| 6 | 16.69 | 4.96 | 38.61 | 7.08 | 1.00 | 1.00 | 50.52 |
| 8 | 11.82 | 3.29 | 29.92 | 5.30 | 1.00 | 1.00 | 38.45 |
| 12 | 6.31 | 2.58 | 20.60 | 3.67 | 1.00 | 1.00 | 26.71 |
| 24 | 5.01 | 1.79 | 12.09 | 2.66 | 1.00 | 1.00 | 16.08 |

TABLE 4
Low concentration standard deviation:

| Time | | | | | | | Total |
|---|---|---|---|---|---|---|---|
| (h) | Vanoxerine | M03 | M04 | M01 | M02 | M05 | Metabolites |
| −15 | 0.00 | 0.00 | 0.00 | 0.00 | 0.00 | 0.00 | 0.00 |
| .5 | 0.00 | 0.00 | 0.00 | 0.00 | 0.00 | 0.00 | 0.00 |
| 1 | 24.47 | 0.00 | 17.68 | 1.67 | 0.00 | 0.98 | 20.45 |
| 2 | 27.50 | 3.10 | 59.32 | 7.56 | 0.13 | 1.05 | 71.04 |
| 3 | 28.16 | 4.18 | 44.96 | 9.05 | 0.00 | 0.58 | 57.77 |
| 4 | 22.66 | 3.28 | 34.95 | 7.06 | 0.00 | 0.46 | 45.53 |
| 6 | 16.11 | 3.72 | 30.77 | 7.28 | 0.00 | 0.16 | 42.04 |
| 8 | 14.20 | 3.51 | 21.42 | 3.71 | 0.00 | 0.00 | 28.30 |
| 12 | 11.19 | 2.27 | 15.60 | 2.86 | 0.00 | 0.00 | 20.34 |
| 24 | 3.07 | 1.69 | 10.44 | 1.72 | 0.00 | 0.00 | 13.40 |

TABLE 5
High concentration group average:

| Time | | | | | | | Total |
|---|---|---|---|---|---|---|---|
| (h) | Vanoxerine | M03 | M04 | M01 | M02 | M05 | Metabolites |
| −15 | 1.00 | 1.00 | 1.00 | 1.00 | 1.00 | 1.00 | 1.00 |
| .5 | 37.67 | 1.06 | 8.71 | 2.55 | 1.00 | 1.19 | 11.01 |
| 1 | 115.12 | 1.98 | 39.82 | 8.64 | 1.00 | 2.10 | 51.33 |
| 2 | 198.71 | 7.96 | 95.46 | 28.00 | 1.05 | 4.51 | 135.79 |
| 3 | 156.49 | 9.98 | 99.70 | 29.64 | 1.03 | 3.64 | 142.69 |
| 4 | 96.14 | 9.83 | 80.45 | 24.93 | 1.02 | 2.01 | 116.64 |
| 6 | 57.08 | 9.03 | 62.44 | 18.08 | 1.00 | 1.55 | 90.10 |
| 8 | 43.18 | 7.37 | 51.08 | 14.50 | 1.00 | 1.52 | 73.46 |
| 12 | 29.30 | 5.93 | 35.57 | 9.98 | 1.00 | 1.13 | 51.52 |
| 24 | 3.07 | 1.69 | 10.44 | 1.72 | 0.00 | 0.00 | 13.40 |

TABLE 6
High concentration group standard deviation:

| Time | | | | | | | Total |
|---|---|---|---|---|---|---|---|
| (h) | Vanoxerine | M03 | M04 | M01 | M02 | M05 | Metabolites |
| −15 | 0.00 | 0.00 | 0.00 | 0.00 | 0.00 | 0.00 | 0.00 |
| .5 | 62.39 | 0.19 | 12.37 | 4.71 | 0.00 | 0.45 | 18.34 |
| 1 | 72.52 | 1.19 | 31.62 | 6.52 | 0.00 | 1.26 | 38.76 |
| 2 | 96.23 | 5.50 | 60.49 | 19.21 | 0.11 | 3.17 | 82.34 |
| 3 | 77.51 | 6.85 | 58.66 | 13.99 | 0.11 | 3.12 | 70.07 |
| 4 | 63.43 | 6.50 | 46.33 | 10.60 | 0.06 | 1.67 | 54.47 |
| 6 | 44.79 | 6.26 | 38.98 | 8.02 | 0.00 | 1.35 | 48.76 |
| 8 | 40.12 | 4.97 | 32.08 | 7.21 | 0.00 | 1.48 | 38.93 |
| 12 | 33.45 | 3.74 | 22.14 | 5.13 | 0.00 | 0.42 | 26.71 |
| 24 | 14.82 | 3.02 | 11.03 | 3.24 | 0.00 | 0.05 | 14.70 |

As can be seen, in Tables 3 and 5, the low concentration group barely has plasma levels rise above 40 ng/ml at any time point in reference to vanoxerine. Whereas, the high concentration group has levels that rise to nearly 200 ng/ml at a time of two (2) hours after administration. Furthermore, the variability with regard to each of the groups is also wider. The standard deviations in Table 4 are lower than those in Table 6, (no T-test or 95% confidence was run), demonstrating that the variability was greater in the high concentration group than the low concentration group.

Accordingly, in view of the data, certain methods may be suitable for normalizing or minimizing the variability with regard to a single dosage of either vanoxerine or one or more of the metabolites thereof through administration with vanoxerine and a P450 inhibitor. What is evident from the examples is the large standard deviations with regard to vanoxerine and certain of the metabolites. Indeed, variability exists not only with vanoxerine but also with the metabolites M01, M02, M03, M04, and M05, generated through the metabolism of vanoxerine. Accordingly, the ability to particularly tailor the administration of vanoxerine based on the metabolic profile of a patient (i.e. fast or slow) provides for an opportunity to improve the treatment on an individual basis. Certain methods may advantageously measure one or more of the metabolites and modification may be intended not simply for modification of vanoxerine but of the metabolites thereof by co-administration with a P450 inhibitor.

Indeed, utilization of P450 inhibitors taken concurrently with vanoxerine allows for appropriate modulation of Cₘₐₓ and tₘₐₓ minimizing variability and increasing efficacy. Therefore, the methods provided for herein, provide for greater accuracy with regard to target plasma levels, thus increasing the safety profile, improving efficacy of treatment, and minimizing side effects that may be associated with treatment.

Although the present invention has been described in considerable detail, those skilled in the art will appreciate that numerous changes and modifications may be made to the embodiments and preferred embodiments of the invention and that such changes and modifications may be made without departing from the spirit of the invention. It is therefore intended that the appended claims cover all equivalent variations as fall within the scope of the invention.

What is claimed is:

1. A pharmaceutical composition for the treatment of cardiac arrhythmia, in unit dosage form comprising vanoxerine, in an amount of from about 20-50% of the composition by weight; a P450 inhibitor from about 1-30% of the composition by weight, a diluent in an amount of from about 20-60% of the composition by weight; a binder in an amount of from about 10-25% of the composition by weight; a disintegrant in an amount of from about 1-5% of the composition by weight; a flowing agent from about 0.2-0.4% of the composition by weight; and a lubricant from about 0.2-0.4% of the composition by weight.

2. The pharmaceutical composition of claim 1, wherein said diluent is lactose monohydrate, wherein said binder is microcrystalline cellulose, wherein said disintegrant is cross-linked sodium carboxymethylcellulose, wherein said flowing agent is colloidal silicon dioxide, and wherein said lubricant is magnesium stearate.

3. The pharmaceutical composition of claim 1, wherein said P450 inhibitor is selected from the group consisting of citrus juice, flavonoids, furanocoumarins, ritanovir, dexamethasone, erythromycin, warfarin, and combinations thereof.

4. A method for maintaining a pre-determined plasma concentration of vanoxerine in a mammal comprising:
a. determining a pre-determined target plasma concentration in the mammal;
b. administering a first dose of vanoxerine to said mammal;
c. measuring the plasma concentration of vanoxerine in the mammal;
d. determining an appropriate subsequent dose of vanoxerine to be taken with a concomitant administration of a P450 inhibitor;
e. administering said subsequent dose of vanoxerine and P450 inhibitor to said mammal.

5. The method of claim 4 wherein the pre-determined plasma level is between about 15-450 ng/ml at a time of between 30 minutes and 4 hours post administration.

6. The method of claim 4 wherein the pre-determined plasma level is between about 40 and 120 ng/ml at a time of between 30 minutes and 4 hours post administration.

7. The method of claim 4 wherein the Cₘₐₓ of the pre-determined plasma level is between about 15-450 ng/ml.

8. A method for administering vanoxerine for treatment of cardiac arrhythmia comprising: administering a first dose of vanoxerine to a patient; measuring the physiological concentration of vanoxerine in the patient; calculating an effective dose of vanoxerine and a P450 inhibitor to modify the calculated plasma level concentration; and administering the effective dose of vanoxerine and P450 inhibitor.

9. The method of claim 8, wherein the P450 inhibitor is selected from the group consisting of a CYP3A4 inhibitor, CYP2C8 inhibitor, CYP2E1 inhibitor, or a CYP2D6 inhibitor, or combinations thereof.

10. The method of claim 8, wherein said P450 inhibitor is selected from the group consisting of citrus juice, flavonoids, furanocoumarins, ritanovir, dexamethasone, erythromycin, warfarin, and combinations thereof.

11. The method of claim 8 further comprising the step of measuring the physiological concentration of one or more metabolites of vanoxerine.

12. The method of claim 8 further comprising a first step of identifying a target plasma level; whereby said calculation of an effective dose of vanoxerine and a P450 inhibitor is calculated to modulate the plasma level concentrations to the target plasma level.

13. A method for maintaining a pre-determined plasma level comprising: administering a first dose of vanoxerine; measuring the physiological concentration of vanoxerine and one or more metabolites; administering a second dosage of vanoxerine in conjunction with a P450 inhibitor; measuring the physiological concentration and one or more metabolites; modifying the dosage of vanoxerine and P450 inhibitor based on the differences between the plasma level after the first administration and the second administration; and administering at least a third dosage of vanoxerine and P450 inhibitor to maintain a pre-determined plasma level.

14. The method of claim 13 wherein the pre-determined plasma level is from about 25 to 125 ng/ml at a time 1 hour after dosing.

15. The method of claim 13 wherein the P450 inhibitor is selected from the group consisting of a CYP3A4 inhibitor, CYP2C8 inhibitor, CYP2E1 inhibitor, or a CYP2D6 inhibitor, or combinations thereof.

16. The method of claim 13, wherein said P450 inhibitor is selected from the group consisting of citrus juice, flavonoids, furanocoumarins, ritanovir, dexamethasone, erythromycin, warfarin, and combinations thereof.

17. A method for modulating plasma level concentrations in a patient being treated for cardiac arrhythmia comprising: administering a first dose of vanoxerine; measuring the physiological concentration of vanoxerine; calculating an effective dose of vanoxerine and a P450 inhibitor to modulate the plasma level concentration; and administering the effective dose of vanoxerine and P450 inhibitor.

18. The method of claim 17 wherein the P450 inhibitor is selected from the group consisting of a CYP3A4 inhibitor, CYP2C8 inhibitor, CYP2E1 inhibitor, or a CYP2D6 inhibitor, or combinations thereof.

19. The method of claim 17, wherein said P450 inhibitor is selected from the group consisting of citrus juice, flavonoids, furanocoumarins, ritanovir, dexamethasone, erythromycin, warfarin, and combinations thereof.

20. The method of claim 17 further comprising a first step of identifying a target plasma level; whereby said calculation of an effective dose of vanoxerine and a P450 inhibitor is calculated to modulate the plasma level concentrations to the target plasma level.
